(19) United States
(12) Patent Application Publication
(10) Pub. No.: US 2026/0015570 A1
(43) Pub. Date: Jan. 15, 2026

(54) Production Method and Culture Device of Cell Tissue

(71) Applicants: SHIMADZU CORPORATION, Kyoto-shi (JP); The University of Osaka, Suita-shi (JP)

(72) Inventors: Masaki KANEDA, Kyoto-shi (JP); Yamato MAEDA, Kyoto-shi (JP); Masayuki OKADA, Kyoto-shi (JP); Hikaru SHIBATA, Kyoto-shi (JP); Koji NUMATA, Kyoto-shi (JP); Rintaro YAMAMOTO, Kyoto-shi (JP); Michiya MATSUSAKI, Suita-shi (JP)

(21) Appl. No.: 19/258,419

(22) Filed: Jul. 2, 2025

(30) Foreign Application Priority Data
Jul. 10, 2024 (JP) 2024-110746

(51) Int. Cl. C12M 1/33; B33Y 10/00; B33Y 30/00; C12M 1/00; C12M 3/00

(52) U.S. Cl. CPC C12M 45/02; B33Y 10/00; B33Y 30/00; C12M 21/08; C12M 47/02

(57) ABSTRACT

The production method includes: filling a soluble liquid holding material that cures under a predetermined condition between a first substrate and a second substrate; curing the filled holding material; and separating at least one cell tissue from at least one of the first substrate and the second substrate by cutting out the cured holding material from the first substrate and the second substrate.

### CROSS REFERENCE TO RELATED APPLICATIONS

This non-provisional application is based on Japanese Patent Application No. 2024-110746 filed on Jul. 10, 2024 with the Japan Patent Office, the entire contents of which are hereby incorporated by reference.

### BACKGROUND OF THE INVENTION

### Field of the Invention

The present disclosure relates to a production method and a culture device of a cell tissue.

### Description of the Background Art

### BACKGROUND DESCRIPTION

Technology to construct cellular tissue by bioprinting has been studied. Research has been progressed to construct cell tissues by bioprinting and using the obtained cell tissues as edible stake meat or processed meat.

Dong-Hee Kang and others developed tendon-gel integrated bioprinting and demonstrated that fibrous cell tissues created by tendon-gel integrated bioprinting can be assembled to construct tissues such as artificial steaks in vitro (Dong-Hee Kang et al., “Engineered whole cut meat-like tissue by the assembly of cell fibers using tendon-gel integrated bioprinting”, Nature Communications volume 12, Article number: 5059 (2021)). The tendon-gel integrated bioprinting disclosed in the above non-patent document is a method in which cells are printed on a gel that mimics a tendon and cultured with both ends of each cell joined to a support that mimics a tendon.

In order to realize such a culturing method, WO 2021/193980 discloses a method of culturing muscle cells by linearly arranging cells in such a manner that both ends of the cells are joined to a support.

WO 2021/193981 discloses a method of culturing cells by linearly printing a bioink that contains cells in a support bath, dissolving gels in the support bath after printing, removing solution in the support bath, and adding a culture solution to culture the cells.

### SUMMARY OF THE INVENTION

By culturing a cell with both ends of the cell supported on a substrate, the cell can be cultured with a tensile force generated between the substrate and the cell. The tensile force facilitates the formation of muscle fibers, muscle tissues or sarcomeric structures, which makes the resulting cell tissue more similar to an animal muscle.

As described above, although it is preferable to culture a cell with both ends of the cell supported on a substrate, it is necessary to remove the substrate when recovering the cell tissue after the culture. However, the cell tissue is very fragile. Therefore, there is a problem that the cell tissue may be damaged when the substrate is removed, which lowers the recovery efficiency.

It is an object of the present disclosure to provide a production method and a culture device of a cell tissue, which are capable of preventing damage to the cell tissue and have good recovery efficiency.

The production method of the present disclosure is a production method of at least one cell tissue. The production method includes: linearly printing at least one cell between a first substrate and a second substrate in such a manner that both ends of the at least one cell are supported on the first substrate and the second substrate, respectively; obtaining the at least one cell tissue by culturing the printed at least one cell; filling a soluble liquid holding material that cures under a predetermined condition between the first substrate and the second substrate; curing the filled holding material; and separating the at least one cell tissue from at least one of the first substrate and the second substrate by cutting out the cured holding material from the at least one of the first substrate and the second substrate.

The culture device of the present disclosure is a bioprinting culture device for culturing at least one cell to obtain at least one cell tissue. The culture device includes: a housing in which a first chamber with a first substrate for supporting the at least one cell disposed therein, a storage chamber configured to store a culture solution, and a second chamber with a second substrate for supporting the at least one cell disposed therein are provided in this order; a replacement unit which replaces the solution stored in the storage chamber with a soluble liquid holding material that cures under a predetermined condition; and a separation unit. The at least one cell is printed linearly from the first chamber toward the second chamber. The separation unit separates the at least one cell tissue stored in the storage chamber from at least one of the first substrate disposed in the first chamber and the second substrate disposed in the second chamber.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic view illustrating a production method up to a culturing step.

FIG. 2 is a schematic view illustrating the other steps subsequent to the culturing step in the production method.

FIG. 3 is a diagram schematically illustrating a structure of a culture device according to an embodiment.

FIG. 4 is a plan view of a wall viewed from a normal direction of a flat surface.

FIG. 5 is a view illustrating a printing step and a separating step using the culture device.

FIG. 6 is a plan view of a wall viewed from a flat surface thereof according to a modification.

FIG. 7 is a diagram schematically illustrating a structure of a culture device according to a modification.

FIG. 8 is a schematic view illustrating a production method according to a modification.

### DESCRIPTION OF THE PREFERRED EMBODIMENTS

Hereinafter, embodiments of the present disclosure will be described in detail with reference to the drawings. In the drawings, the same or corresponding parts are denoted by the same reference numerals, and the description thereof will not be repeated.

### [Production Method of Cell Tissue]

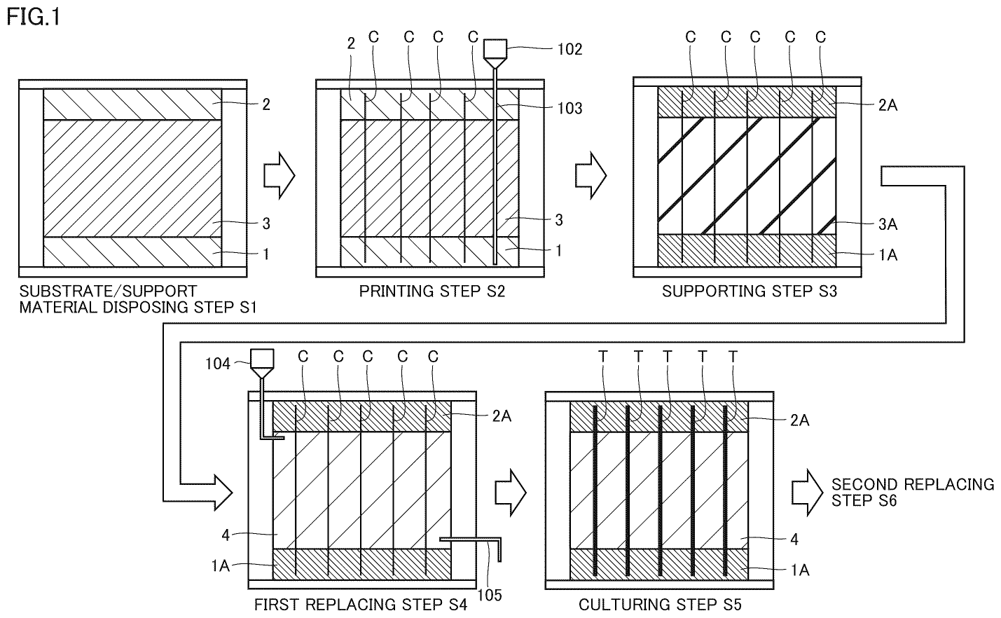

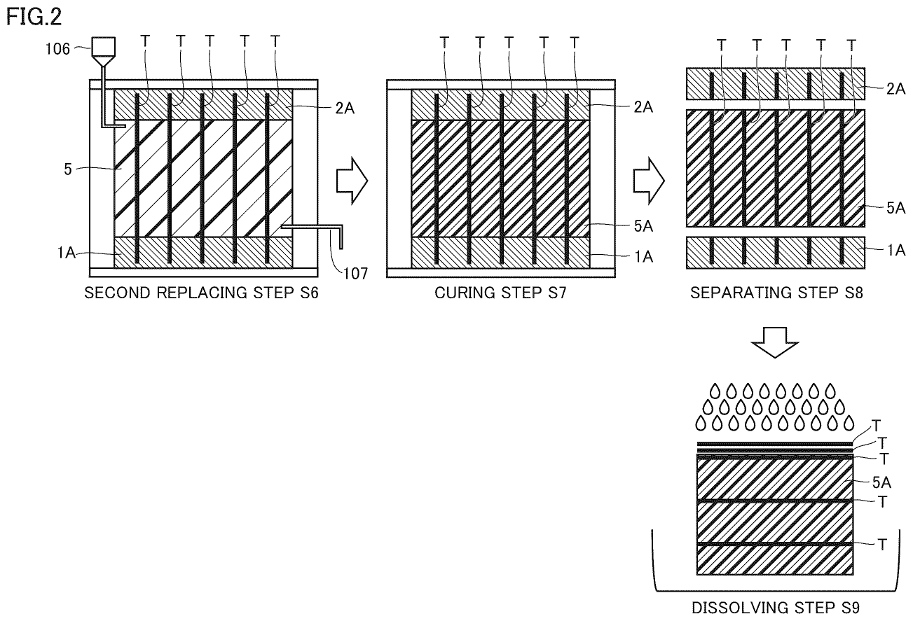

A production method will be described with reference to FIGS. 1 and 2. FIG. 1 is a schematic view illustrating a production method up to a culturing step. FIG. 2 is a schematic view illustrating the other steps subsequent to the culturing step in the production method.

The cell tissue is obtained by culturing a cell linearly printed. The cell is not particularly limited as long as it is linearly printed and cultured to form a fibrous cell tissue. The cell may be, for example, a cell derived from an animal, a cell derived from a human, or a cell derived from an animal other than a human. The cell used in the present embodiment is, for example, a skeletal muscle cell. The skeletal muscle cell may be a muscle-derived cell or a stem cell-derived cell.

With reference to FIGS. 1 and 2, the production method according to the present embodiment includes a substrate/support material disposing step S1, a printing step S2, a supporting step S3, a first replacing step S4, a culturing step S5, a second replacing step S6, a curing step S7, a separating step S8, and a dissolving step S9.

### (Substrate/Support Material Disposing Step S1)

A substrate 1, a support material 3, and a substrate 2 are disposed in a culture device in this order. In other words, support material 3 is sandwiched between substrate 1 and substrate 2.

Substrates 1 and 2 may be any substrate that binds to a cell to be printed in the printing step S2 to support the cell, and may be made of a material appropriately selected by those skilled in the art. Substrates 1 and 2 may be either a solid or a liquid. The solid state includes a gel state. The physical properties of substrates 1 and 2 are not particularly limited, and for example, substrates 1 and 2 may have such a physical property that the state thereof changes under a predetermined condition or may such a physical property that the state thereof does not change under a predetermined condition. The predetermined condition is, for example, temperature, pressure, electrical stimulus, pH, or the like. Substrates 1 and 2 are, for example, collagen or collagen nanofibers. The type of collagen is not particularly limited. For example, substrates 1 and 2 may be formed from a plurality of different types of collagen. The composition of substrate 1 and the composition of substrate 2 may be different from each other. In the present embodiment, substrates 1 and 2 will be described as collagen nanofibers.

Support material 3 is made of a dissolvable material. For example, support material 3 is a solution obtained by dissolving a macromolecular substance such as gelatin, agar, or gellan gum in an aqueous solvent. In this case, in the substrate/support material disposing step S1, support material 3 may be in a gel form or a sol form. Support material 3 may have a thixotropy property, which means that when a force is applied, the viscosity thereof decreases and becomes a liquid, and when the force is removed, the viscosity thereof gradually recovers. Support material 3 may be, for example, a mixed solution prepared by pulverizing a gelled sample and dispersing the pulverized sample in a solvent such as a liquid medium. The gelled sample is prepared, for example, by dissolving a macromolecular substance such as gelatin, agar, or gellan gum in an aqueous solvent to form a gel. In the substrate/support material disposing step S1, support material 3 is described as a mixed solution in which a sample obtained by gelling a gelatin solution is pulverized and dispersed in a liquid medium.

In the present embodiment, as an example, after substrate 1 is disposed in liquid form, substrate 1 is partially cured to increase the viscosity, support material 3 made of a mixed solution which is obtained by dispersing a gelled sample in a solvent is disposed on substrate 1. Thereafter, substrate 2 is disposed in liquid form on support material 3. The method of disposing each sample is not particularly limited.

### (Printing Step S2)

The printing step S2 is a step of linearly printing cells C. At this time, cells C are printed in such a manner that one end of each cell C is disposed on substrate 1 and the other end thereof is disposed on substrate 2. For example, cells C are printed by the following method. A bioink containing cells C is filled into a syringe 102, the tip of which is attached with a nozzle 103. Thereafter, nozzle 103 is inserted from substrate 2 toward substrate 1 until the tip of nozzle 103 is located in substrate 1. Cells C are linearly printed from substrate 1 toward substrate 2 by ejecting the bioink from nozzle 103 while moving the tip of nozzle 103 in the direction from substrate 1 toward substrate 2.

The printing of cells is performed in support material 3. When support material 3 has a thixotropy property, the viscosity thereof decreases due to a stress caused by the movement of nozzle 103 and the ejection of the bioink during the printing, and the viscosity thereof recovers after the printing. Since the viscosity decreases during the printing, the nozzle can be easily moved and the bioink can be easily ejected. On the other hand, since the viscosity recovers and increases after the printing, support material 3 maintains the shape of the printed cells C and protects the printed cells C. When support material 3 is a solution obtained by dissolving a macromolecular substance in an aqueous solvent, the printing may be performed when support material 3 is in a sol form, and support material 3 may be turned into a gel form after the printing.

The printing step S2 may be performed automatically or semi-automatically using a known three-dimensional bioprinting apparatus. Alternatively, a multi-nozzle dispenser having a plurality of nozzles may be used to print a plurality of cells at one time.

The number of cells C linearly printed in the printing step S2 is not particularly limited as long as it is 1 or more, and may be 2 or more, 10 or more, 100 or more, 1000 or more, or 10000 or more, which may be appropriately selected according to the size of the final product, the culture device, and the like.

The diameter of a line formed by printing cells C is not particularly limited, and may be appropriately selected depending on the type of cells C, the viscosity of the bioink, the cell tissue to be obtained, the diameter of the usable nozzle 103, or the like. For example, the diameter of the line may be 100 μm or less, 100 μm or more, 1 mm or less, 1 mm or more, 10 mm or less, 10 mm or more, 100 mm or less, or 100 mm or more.

In the present embodiment, a plurality of cells C may be printed linearly from substrate 1 toward substrate 2, and the extending directions of the plurality of cells C may be or may not be parallel to each other. In the present embodiment, the plurality of cells C are printed in such a manner that the extending directions of the plurality of cells C are parallel to each other.

### (Supporting Step S3)

The supporting step S3 is a step of curing substrates 1 and 2 into substrates 1A and 2A to support both ends of each cell C on substrates 1A and 2A, respectively. In FIGS. 1 and 2, in order to clearly show that the state of substrates 1 and 2 has been changed from the liquid state to the solid state, the liquid substrates are represented as substrates 1 and 2, and the solid substrates are represented as substrates 1A and 2A with a different reference numeral. The solid state includes a gel state, and curing substrates 1 and 2 means transforming substrates 1 and 2 into a state suitable for supporting both ends of each cell C.

Substrates 1A and 2A may have a reversible property, which means that the state of the substrate can change from a solid state to a liquid state, or may have an irreversible property, which means that the state of the substrate cannot change from a solid state to a liquid state.

When the state of substrates 1 and 2 gradually changes from a liquid state to a solid state, the printing step S2 and the supporting step S3 may be performed in parallel. In other words, substrates 1 and 2 may be cured while cells C are being printed. By performing the printing step S2 and the supporting step S3 in parallel, at least one cell C is linearly printed with both ends thereof supported on substrates 1 and 2, respectively.

The solid (gel) in support material 3 may be transferred to a liquid (sol) while substrates 1 and 2 are being cured. For example, in the case where substrates 1 and 2 do not dissolve under a condition where the solid (gel) in support material 3 is transferred to the liquid (sol), the solid (gel) in support material 3 can be transferred to the liquid (sol) while substrates 1 and 2 are being cured by controlling the culture device so as to satisfy the condition for transferring the solid (gel) in support material 3 to the liquid (sol) and the condition for curing substrates 1 and 2. By transferring the solid (gel) in support material 3 to the liquid (sol), support material 3 can be easily replaced with the culture solution in the following first replacement step S4. In FIG. 1, the support material that contains a solid (gel) is illustrated as support material 3, and the support material after being transferred to a liquid (sol) is illustrated as support material 3A with a different reference numeral.

In the present embodiment, substrates 1 and 2 are collagen nanofibers, and support material 3 is a mixed solution in which a sample obtained by gelling a gelatin solution is pulverized and dispersed in a liquid medium. By maintaining the temperature in the culture device lower than a temperature at which collagen does not denature and equal to or higher than a temperature at which gelatin in support material 3 is transferred to a sol, the gel in support material 3 can be transferred to a sol while substrates 1 and 2 are being cured. For example, the temperature in the culture device may be maintained less than about 40° C., at 25° C. or more, at 35° C. or more, or at 37° C. or more. If the temperature in the culture device is maintained in such a temperature range, gelatin in support material 3 is dissolved while substrates 1 and 2 are being cured. In consideration of the influence on the growth of cells C, the temperature in the culture device is preferably maintained at 30° C. to 40° C., more specifically at 37° C.

### (First Replacing Step S4)

The first replacing step S4 is a step of replacing support material 3A with a culture solution 4. For example, culture solution 4 is injected between substrates 1A and 2A from a syringe 104 to eject the sample (support material 3A) between substrates 1 and 2 from a waste solution port 105. At this time, support material 3A can be easily removed by transferring the solid (gel) in support material 3 to a liquid (sol) in advance in the supporting step S3.

Culture solution 4 may be appropriately selected by those skilled in the art according to the type of cells or the like. For example, culture solution 4 may contain a differentiation-inducing factor for inducing differentiation of cells.

Although the supporting step S3 and the step of dissolving support material 3A are performed in parallel, they may be performed independently. For example, the step of dissolving support material 3A may be performed after the supporting step S3, and then the first replacing step S4 may be performed.

### (Culturing Step S5)

The culturing step S5 is a step of culturing cells C in culture solution 4 to obtain cell tissues T. The culture conditions may be appropriately designed by those skilled in the art according to the type of cells and the type of cell tissues to be obtained. During the culturing step S5, the first replacing step S4 may be performed to change the culture solution.

### (Second Replacing Step S6)

With reference to FIG. 2, the second replacing step S6 is a step of replacing culture solution 4 with a holding material 5 that is a soluble liquid cured under a predetermined condition and filling holding material 5 between substrate 1 and substrate 2. For example, holding material 5 is injected between substrates 1A and 2A from a syringe 106 to eject culture solution 4 from a waste solution port 107.

Holding material 5 is made of a soluble material, and is treated as a dissolved liquid in the second replacing step S6. Holding material 5 is obtained by dissolving a macromolecular substance such as gelatin, agar, or gellan gum in an aqueous solvent. In the present embodiment, holding material 5 is obtained by dissolving gelatin in an aqueous solvent. It is preferable that holding material 5 has such a hardness that holding material 5 can hold cell tissues T when cured. For example, when gelatin having a jelly strength of 300 g is used, holding material 5 is a gelatin solution in which the gelatin content is adjusted to a range of 35 mg/mL to 45 mg/mL, and more specifically, a gelatin solution in which the gelatin content is adjusted to 40 mg/mL.

Support material 3 and holding material 5 are common in that each is a sample that contains a macromolecular substance. In the present embodiment, support material 3 is a solution in which a macromolecular substance is gelled and dispersed in a solvent, whereas holding material 5 is a solution in which a macromolecular substance is completely dissolved and converted into a sol.

Holding material 5 may be used as a culture solution to be used in the culturing step S5. For example, a macromolecular substance such as gelatin may be dissolved in culture solution 4, and the obtained solution may be used to culture cells C. In this case, it is not necessary to perform the second replacing step S6. In other words, the step of filling holding material 5 may be performed before the culturing step S5 or in parallel with the culturing step S5. In the second replacing step S6, culture solution 4 is replaced with holding material 5, but holding material 5 may be used to replace a solution different from culture solution 4. In other words, a pretreatment step of increasing the strength of cell tissues T may be performed, or a step of washing cell tissues T may be performed before the second replacing step S6.

### (Curing Step S7)

The curing step S7 is a step of curing holding material 5. In FIG. 2, in order to clearly show that the state of holding material 5 has been changed from the liquid state to the solid state, the liquid holding material is represented as holding material 5, and the solid holding material is represented as holding material 5A with a different reference numeral. For example, the temperature in the culture device is maintained at 20° C. or less, 10° C. or less, or 5° C. or less. Since the curing condition varies depending on the type of holding material 5, the curing condition is determined according to the type of holding material 5.

### (Separating Step S8)

The separating step S8 is a step of separating substrates 1A and 2A from cell tissues T by separating the cured holding material 5A from the cured substrates 1A and 2A. In this step, cell tissues T are separated from substrates 1A and 2A while being protected by holding material 5A. Therefore, a tensile force is less likely to be applied to cell tissues T in the separating step, and damage to cell tissues T can be prevented. Thus, the recovery rate of cell tissues T can be increased.

In the present embodiment, the separating step S8 may be performed by taking out the entire block including substrates 1A and 2A and holding material 5A from a culture device 100. Even by taking out the entire block, since cell tissues T are protected by holding material 5A, damage to cell tissues T can be prevented, and the recovery rate of cell tissues T can be increased.

### (Dissolving Step S9)

The dissolving step S9 is a step of dissolving holding material 5A. Thus, cell tissues T can be recovered. Since the dissolving condition varies depending on the type of holding material 5A, the dissolving condition is determined according to the type of holding material 5A. For example, when holding material 5A is dissolved by temperature, holding material 5A that contains cell tissues T may be dissolved by applying water or the like having a predetermined temperature to holding material 5A to recover cell tissues T. When holding material 5A is dissolved by pH, holding material 5A may be dissolved by applying a solution (for example, a buffer solution) having a predetermined pH to holding material 5A to recover cell tissues T.

More specifically, when holding material 5A is made of gelatin, water having a temperature of less than 40° C., 25° C. or more, 35° C. or more, or 37° C. or more is applied to holding material 5A. When holding material 5A is made of gellan gum, a cationic solution such as a tris-hydrochloric acid buffer solution, a tris-maleic acid buffer solution, or a bis-tris-buffer solution is applied to holding material 5A. When holding material 5A is made of agar, water having a temperature of 80° C. or more is applied to holding material 5A.

In consideration of the influence on cell tissues T, holding material 5A is preferably made of a material that dissolves at a temperature of less than 40° C. or a material that dissolves at a pH of 6.8 to 7.2. However, if holding material 5 can be separated from cell tissues T at the same time as holding material 5A is dissolved, the influence on cell tissues T can be reduced. Therefore, holding material 5 is not limited to those that dissolve under the above-described conditions, but may also dissolve under conditions that affect cell tissues T.

In the present embodiment, a plurality of cells C are printed in the printing step S2 to obtain a plurality of cell tissues T. Each of the plurality of cells C is linearly printed from substrate 1 toward substrate 2. Therefore, the plurality of cell tissues T are held by holding material 5A in such a manner that the extending directions thereof are aligned with each other. Therefore, as illustrated in FIG. 2, at the time of dissolving holding material 5A, it is preferable to dispose holding material 5A so that a surface on which holding material 5A is disposed and the extending direction of cell tissues T are aligned with each other. Thus, the plurality of cell tissues T can be recovered in such a manner that the extending directions are aligned with each other. As a result, in the step of assembling the recovered cell tissues T, the step of aligning the fiber directions of cell tissues T becomes unnecessary.

According to the present embodiment, in the separating step S8, both of substrates 1A and 2A are separated from cell tissues T. However, in the separating step S8, at least one of substrates 1A and 2A may be separated from substrates 1A and 2A. When a material that does not dissolve in the dissolving step S9 is used as the substrate, since one end of each cell tissue T is held even after the dissolving step S9, the plurality of cell tissues T may be easily handled together.

[Culture Device for Implementing Separating step]

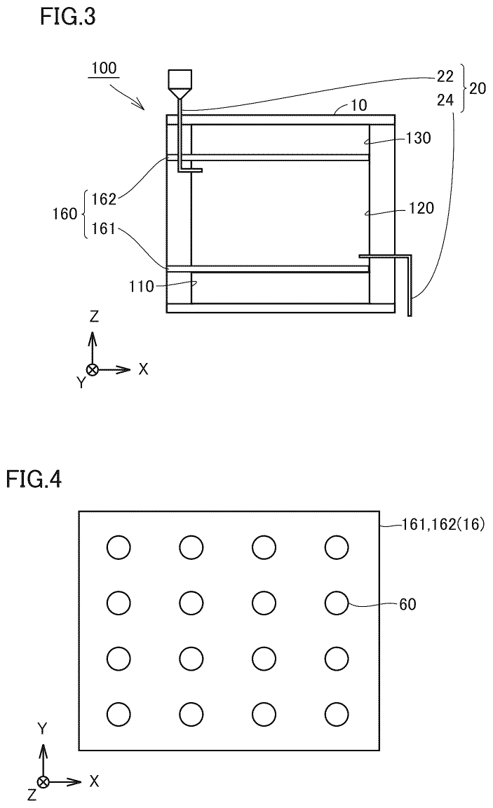

FIG. 3 is a diagram schematically illustrating a structure of a culture device 100 according to an embodiment. Culture device 100 is a bioprinting culture device for culturing cells to obtain cell tissues. Culture device 100 includes a housing 10, a replacement unit 20, and a separation unit 160.

In housing 10, a first chamber 110 with substrate 1 disposed therein, a storage chamber 120 configured to store culture solution 4, and a second chamber 130 with substrate 2 disposed therein are provided in this order.

The substrate is disposed in each of first chamber 110 and second chamber 130. Culture device 100 may be configured in such a manner that the substrate is preliminarily disposed in each of first chamber 110 and second chamber 130, or may be configured in such a manner that the substrate can be inserted and disposed in each of first chamber 110 and second chamber 130 later.

Replacement unit 20 replaces the sample (solution) filled in storage chamber 120 with another sample (solution). Specifically, replacement unit 20 includes an insertion port 22 for inserting a tube, a syringe, or the like to which a tube pump is attached, and a discharge port 24 for discharging the sample in storage chamber 120 to the outside of culture device 100. Insertion port 22 and discharge port 24 are each connected to storage chamber 120.

For example, in the first replacing step S4, replacement unit 20 replaces the sample filled in storage chamber 120 from support material 3A to culture solution 4. In addition, after cells C are cultured in the culturing step S5 and thereby cell tissues T are obtained, replacement unit 20 replaces the sample filled in storage chamber 120 from culture solution 4 to holding material 5 in the second replacing step S6.

Separation unit 160 is a device for separating cell tissues T obtained by culturing cells C in storage chamber 120 from the substrate. Separation unit 160 includes a first wall 161 and a second wall 162. First wall 161 is a wall that partitions first chamber 110 and storage chamber 120 from each other, and is disposed in housing 10. Second wall 162 is a wall that partitions storage chamber 120 and second chamber 130 from each other, and is disposed in housing 10. First wall 161 is disposed in housing 10 in such a manner that a flat surface of first wall 161 faces first chamber 110. Second wall 162 is disposed in housing 10 in such a manner that a flat surface of second wall 162 faces second chamber 130.

Both first wall 161 and second wall 162 are slidable. More specifically, first wall 161 is slidable along a flat surface of first wall 161. Second wall 162 is slidable along a flat surface of second wall 162. In FIG. 3, a sliding direction is defined as the X axis, a normal direction of the flat surface is defined as the Z axis, and an axis orthogonal to the X axis and the Z axis is defined as the Y axis. In the present embodiment, first wall 161 and second wall 162 are disposed in parallel to each other in housing 10, and have the same sliding direction. The sliding direction of first wall 161 and the sliding direction of second wall 162 may be different from each other.

When a substrate is disposed in each of first chamber 110 and second chamber 130, it can be said that first wall 161 is disposed in culture device 100 in such a manner that the flat surface thereof faces the substrate. Similarly, it can be said that second wall 162 is disposed in culture device 100 in such a manner that the flat surface thereof faces the substrate.

FIG. 4 is a plan view of a wall viewed from a normal direction of a flat surface. Hereinafter, first wall 161 and second wall 162 are collectively referred to as “wall 16”. An opening 60 is formed in wall 16. The plan view illustrated in FIG. 4 is obtained by viewing wall 16 from the normal direction (Z-axis direction) of the flat surface of wall 16.

At least one opening 60 is formed in wall 16. The number of openings 60 is not particularly limited, and the number may be 2 or more, 10 or more, 100 or more, 1000 or more, or 10000 or more. Since at least one opening 60 is formed in first wall 161 and second wall 162, the printing step S2 can be performed without removing first wall 161 and second wall 162.

First wall 161 and second wall 162 each may be a plate-shaped member in which opening 60 is not formed. In this case, after substrates 1 and 2 and support material 3 are disposed in culture device 100, the printing step may be performed by sliding first wall 161 and second wall 162 to eliminate the separation between storage chamber 120 and first chamber 110 and the separation between storage chamber 120 and second chamber 130. Thereafter, in the separating step, cell tissues T may be separated from substrates 1 and 2 by sliding first wall 161 and second wall 162 between storage chamber 120 and first and second chambers 110, 130.

The size of opening 60 should be large enough to allow a nozzle to pass through to eject the bioink containing the cells. When opening 60 is circular, the diameter of opening 60 may be 100 μm or less, 100 μm or more, 1 mm or less, 1 mm or more, 10 mm or less, 10 mm or more, 100 mm or less, or 100 mm or more.

First wall 161 and second wall 162 are preferably disposed in culture device 100 in such a manner that opening 60 formed in first wall 161 and opening 60 formed in second wall 162 face each other.

As illustrated in FIG. 4, wall 16 may be formed with a plurality of openings 60 arranged in two-dimensions. Cells C are printed in such a manner that at least one cell C passes through each of the plurality of openings 60 arranged in two-dimensions. As described above, since wall 16 is formed with a plurality of openings 60 arranged in two-dimensions, each cell tissue T can be easily handled after cell tissues T are separated from substrates 1 and 2.

The plurality of openings 60 may be arranged in two directions intersecting each other. The intersection angle is 30° or less, 30° or more, 45° or less, 45° or more, 90° or less, or 90° or more. In the example illustrated in FIG. 4, the plurality of openings 60 are arranged in two directions orthogonal to each other. The plurality of openings 60 may be arranged in a staggered grid pattern.

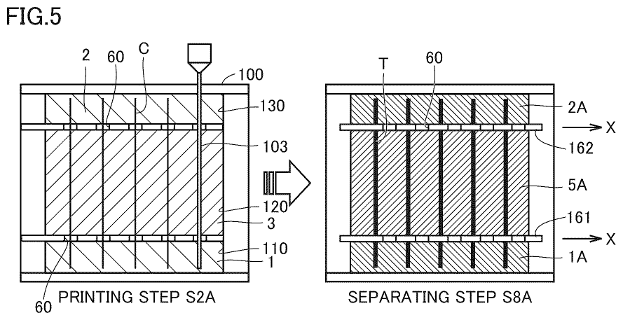

FIG. 5 is a view illustrating a printing step and a separating step using the culture device. In the printing step S2A, cells C can be linearly arranged in culture device 100 in such a manner that both ends of each cell C are located in first chamber 110 and second chamber 130, respectively, by moving nozzle 103 to eject the bioink containing cells C as described below. First, nozzle 103 is disposed to pass through opening 60 of second wall 162 and opening 60 of first wall 161. Next, nozzle 103 is moved so that the tip of nozzle 103 passes through opening 60 of first wall 161 and opening 60 of second wall 162 while the bioink is being ejected from nozzle 103. Thus, cells C can be linearly arranged to pass through opening 60 of second wall 162 from opening 60 of first wall 161. As a result, cells C are linearly arranged from substrate 1 disposed in first chamber 110 toward substrate 2 disposed in second chamber 130. Thereafter, both ends of each linear cell C can be supported on substrates 1 and 2 by curing substrates 1 and 2.

Next, in the separating step S8A, first wall 161 and second wall 162 are slid in the X-axis direction along the corresponding flat surface. Thus, cell tissues T, which are arranged to pass through opening 60, are cut by an edge of opening 60. As a result, cell tissues T are separated from substrates 1A and 2A.

Thus, since cell tissues T can be separated from substrates 1 and 2 only by sliding first wall 161 and second wall 162, respectively, the separation operation can be easily performed.

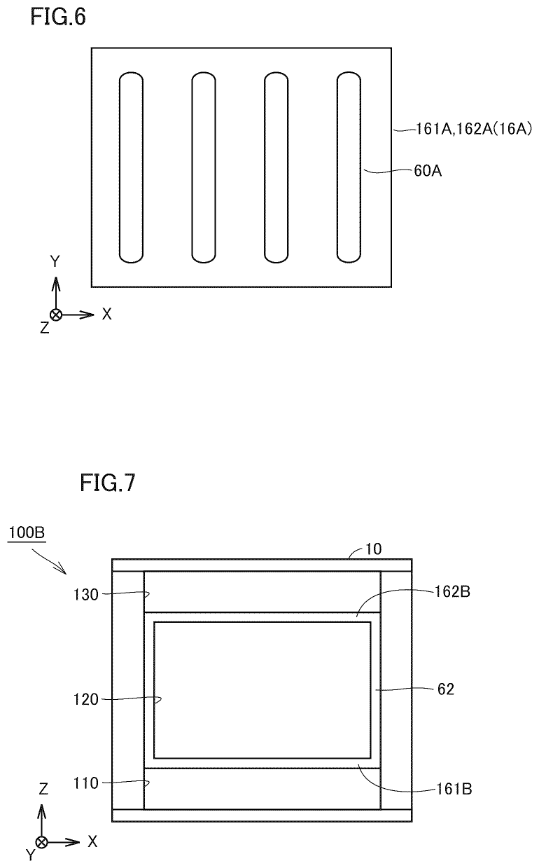

The arrangement pattern and shape of opening 60 are not limited to those illustrated in FIG. 4. FIG. 6 is a plan view of a wall viewed from a flat surface thereof according to a modification. A wall 16A is formed with a plurality of openings 60A, each of which has an elongated shape in which a length along the X-axis direction (sliding direction) is shorter than a length along the Y-axis direction orthogonal to the X-axis direction (sliding direction).

When each opening 60A has an elongated shape, as compared with the case where a plurality of openings 60 are formed in the column direction, it is easy to perform alignment when a plurality of nozzles such as a multi-nozzle dispenser are inserted into culture device 100 at a time. Since the length along the X-axis direction is shorter than the length along the Y-axis direction, the distance from cell tissues T to the edge of each opening 60A can be reduced. Thus, it is possible to shorten the distance to slide wall 16A to cut cell tissues T from the substrates.

In addition, although it is described that storage chamber 120 is divided into first chamber 110 and second chamber 130 by first wall 161 and second wall 162, storage chamber 120 may be formed by arranging a box in housing 10. FIG. 7 is a diagram schematically illustrating a structure of a culture device according to a modification. A culture device 100B may include a box 62 disposed in such a manner that the box is sandwiched between predefined spaces (first chamber 110 and second chamber 130) in housing 10. The inner space of box 62 corresponds to storage chamber 120. A first wall 161B which forms a boundary between first chamber 110 and box 62 is formed with an opening, and a second wall 162B which forms a boundary between second chamber 130 and box 62 is formed with an opening. The shape of the opening is not particularly limited, and for example, the opening having the shape illustrated in FIG. 4 or FIG. 6 may be formed in first wall 161B and second wall 162B.

Each of first wall 161B and second wall 162B is slidable along the flat surface of the wall, and at least one of first wall 161B and second wall 162B is openable in the Z-axis direction perpendicular to the wall.

Thus, by configuring at least one of first wall 161B and second wall 162B to be openable and closable like a lid, it is possible to easily take out the holding material containing the cell tissues from culture device 100B after the cell tissues are separated from the substrates. Thus, each cell tissue can be easily handled after the cell tissues are separated from the substrates.

Although first wall 161 and second wall 162 that partition a respective space in housing 10 are defined as separation unit 160, the configuration of separation unit 160 is not limited thereto. For example, separation unit 160 may be realized by providing an opening in a side surface of housing 10 so that a flat plate can be inserted into storage chamber 120 in a direction (the X-axis direction or the Y-axis direction) orthogonal to the extending direction (the Z-axis direction) of cell tissues T. In this case, first wall 161 and second wall 162 may not be provided.

### Modification

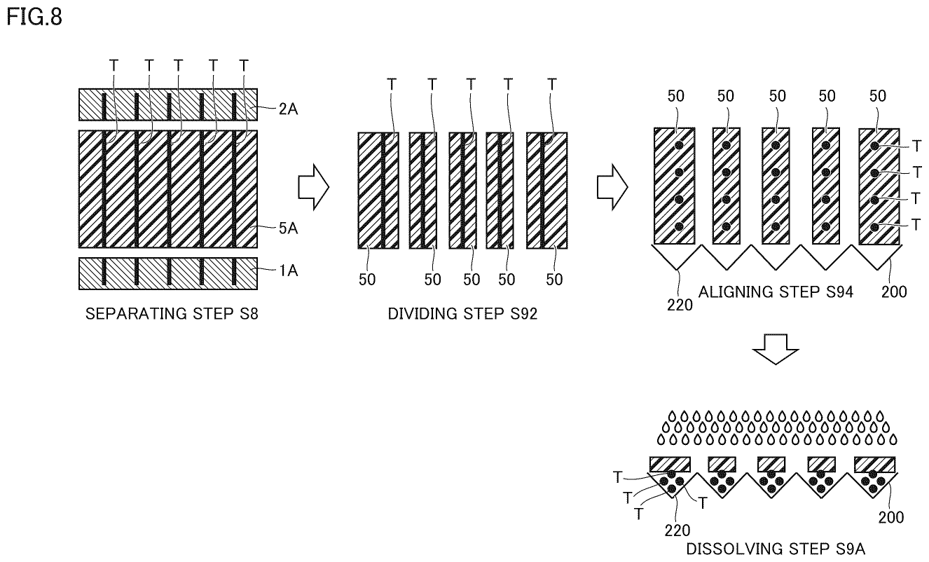

In the above-described embodiment, after cell tissues T are separated from substrates 1A and 2A, holding material 5A is dissolved in the dissolving step S9 as a large mass. However, before holding material 5A is dissolved, holding material 5A may be divided into a plurality of blocks and then the plurality of blocks may be dissolved. FIG. 8 is a schematic view illustrating a production method according to a modification. Since the production method according to the modification has the same steps up to the separating step as the production method illustrated in FIGS. 1 and 2, only the steps after the separating step are illustrated in FIG. 8.

The production method according to the modification is different from the production method illustrated in FIGS. 1 and 2 in that the production method according to the modification further includes a dividing step S92 and an aligning step S94, and includes a dissolving step S9A instead of the dissolving step S9.

### (Dividing Step S92)

In the dividing step S92, the cured holding material 5A is divided into a plurality of blocks 50 along the extending direction of cell tissues T. At least one block 50 of the plurality of blocks 50 includes at least one cell tissue T. The number of cell tissues T to be included in each block 50 may be appropriately selected by those skilled in the art, and is determined according to, for example, subsequent processing steps. The number of blocks 50 may be two or more. For example, when a plurality of cells C are printed in two directions orthogonal to each other as illustrated in FIG. 4, holding material 5A may be divided into one row or two rows or more.

Since holding material 5A is divided into a plurality of blocks 50, it is possible to handle the cell tissues in each block 50, and since the cell tissues are protected by the cured holding material 5A constituting the plurality of blocks 50, it is easy to handle the cell tissues. In addition, one or a bundle of cell tissues T can be easily obtained by dissolving each block 50. In addition, in a case where an operation of combining a plurality of types of cell tissues T is performed to create an aggregate that mimics the structure of a specific tissue, it is easier to perform the combination operation in each block 50 than in a case where the combination operation is performed on each cell tissue T.

### (Aligning Step S94)

The aligning step S94 is a step of aligning the plurality of blocks 50. The plurality of blocks 50 are arranged so that the extending directions of cell tissues T are aligned with each other. In this case, each of the plurality of blocks 50 may be arranged on a net 200 having a wavy structure. When each block 50 is arranged on net 200 having a wavy structure, each block 50 may be disposed in a recess 220 so that the extending direction of recess 220 is aligned with the extending direction of cell tissues T included in each block 50.

### (Dissolving Step S9A)

The dissolving step S9A is performed after the aligning step S94. A plurality of cell tissues T can be obtained in such a manner that the extending directions thereof are aligned with each other by dissolving holding material 5A after the plurality of blocks 50 are disposed in such a manner that the extending directions of cell tissues T are aligned with each other. Therefore, it is not necessary to align the extending directions of cell tissues T with a tweezer or the like, and thereby the risk of damaging cell tissues T can be reduced. Since the dissolution conditions are the same as those of the above-described embodiment, the description thereof will not be repeated.

In addition, when the plurality of blocks 50 are disposed on net 200 having a wavy structure and then dissolved, cell tissues T can be collected in recess 220, and thereby a bundle of cell tissues T can be easily obtained.

### ASPECTS

It will be understood by those skilled in the art that the embodiments described above are specific examples of the following aspects.

(First Aspect) The production method according to one aspect is a production method of at least one cell tissue. The production method includes: linearly printing the at least one cell between a first substrate and a second substrate in such a manner that both ends of the at least one cell are supported on the first substrate and the second substrate, respectively; obtaining the at least one cell tissue by culturing the printed at least one cell; filling a soluble liquid holding material that cures under a predetermined condition between the first substrate and the second substrate; curing the filled holding material; and separating the at least one cell tissue from at least one of the first substrate and the second substrate by cutting out the cured holding material from the at least one of the first substrate and the second substrate.

According to the production method described in the first aspect, by separating the cell tissues from the first substrate and the second substrate with the cell tissues held by the cured holding material, it is possible to prevent damage to the cell tissues and improve the recovery efficiency of the cell tissues.

(Second Aspect) In the production method according to the first aspect, a first member having a flat plate shape and a second member having a flat plate shape may be disposed between the first substrate and the second substrate in this order. A flat surface of the first member is disposed to face the first substrate, and a flat surface of the second member is disposed to face the second substrate. The first member is slidable along the flat surface of the first member. The second member is slidable along the flat surface of the second member. The separating includes separating the at least one cell tissue from the first substrate and the second substrate by sliding the first member and the second member.

According to the production method described in the second aspect, since the cell tissues can be separated from the substrates only by sliding the first member and the second member, the separation operation can be easily performed.

(Third Aspect) In the production method according to the second aspect, the first member is formed with at least one first opening through which the at least one cell passes. The second member is formed with at least one second opening through which the at least one cell passes. The printing includes printing the at least one cell through the at least one first opening and the at least one second opening.

According to the production method described in the third aspect, since each of the first member and the second member is formed with an opening, cells can be printed without removing the first member and the second member.

(Fourth Aspect) In the production method according to any one of the first to third aspects, the printing includes individually and linearly printing a plurality of cells. The obtaining of the at least one cell tissue includes obtaining a plurality of cell tissues by culturing the plurality of cells. The production method further includes obtaining a plurality of blocks by dividing the holding material along an extending direction of the plurality of cell tissues held by the cured holding material.

According to the production method described in the fourth aspect, since the cell tissues can be handled in each block and the cell tissues are protected by the cured holding material constituting the plurality of blocks, the cell tissues can be easily handled. In addition, in a case where an operation of combining a plurality of types of cell tissues is performed to create an aggregate that mimics the structure of a specific tissue, it is easier to perform the combination operation in each block than in a case where the combination operation is performed on each cell tissue.

(Fifth Aspect) The production method according to any one of Items 1 to 4 further includes dissolving the cut-out holding material.

According to the production method described in the fifth aspect, the cell tissues can be recovered simply by dissolving the cut-out holding material.

(Sixth Aspect) The production method according to the fourth aspect further includes: disposing the plurality of blocks in such a manner that the extending directions of the plurality of cell tissues are aligned; and dissolving the holding material constituting each of the plurality of disposed blocks.

According to the production method described in the sixth aspect, a plurality of cell tissues can be obtained in such a manner that the extending directions are aligned with each other by dissolving the blocks disposed in such a manner that the extending directions of the cell tissues are aligned with each other. Therefore, it is not necessary to align the extending directions of the cell tissues with a tweezer or the like, and thereby the risk of damaging the cell tissue can be reduced.

(Seventh Aspect) In the production method according to any one of the first to sixth aspects, the filling of the holding material is performed after the at least one cell tissue is obtained.

(Eighth Aspect) A culture device according to one aspect is a bioprinting culture device for culturing at least one cell to obtain at least one cell tissue. The culture device includes: a housing in which a first chamber with a first substrate for supporting the at least one cell disposed therein, a storage chamber configured to store a culture solution, and a second chamber with a second substrate for supporting the at least one cell disposed therein are provided in this order; a replacement unit which replaces the solution stored in the storage chamber with a soluble liquid holding material that cures under a predetermined condition; and a separation unit. The at least one cell is linearly printed from the first chamber toward the second chamber. The separation unit separates the at least one cell tissue stored in the storage chamber from at least one of the first substrate disposed in the first chamber and the second substrate disposed in the second chamber.

According to the culture device described in the eighth aspect, since the holding material that is cured under a predetermined condition can be filled into the storage chamber by the replacement unit, the cell tissues can be cut off from the substrates in such a manner that each of the cell tissues is held by the cured holding material. As a result, damage to the cell tissues can be prevented, and the recovery efficiency of the cell tissues can be improved.

(Ninth Aspect) In the culture device according to the eighth aspect, the separation unit includes a first wall that is disposed in the housing to partition the first chamber and the storage chamber from each other, and a second wall that is disposed in the housing to partition the second chamber and the storage chamber from each other. The first wall is slidable along a flat surface of the first wall. The second wall is slidable along a flat surface of the second wall. The separation unit is configured to separate the first substrate and the second substrate from each of the at least one cell tissue by sliding the first wall and the second wall.

According to the culture device of the ninth aspect, since the cell tissues can be separated from the substrates only by sliding the first wall and the second wall, the separation operation can be easily performed.

(Tenth Aspect) In the culture device according to the ninth aspect, the first wall is formed with at least one first opening through which the at least one cell passes. The second wall is formed with at least one second opening through which the at least one cell passes.

According to the culture device of the tenth aspect, the cells can be linearly printed without removing the first wall and the second wall.

(Eleventh Aspect) In the culture device according to the tenth aspect, the first wall is formed with a plurality of first openings arranged in two dimensions when the first wall is viewed from a normal direction of a flat surface of the first wall. The second wall is formed with a plurality of second openings arranged in two dimensions when the second wall is viewed from a normal direction of a flat surface of the second wall.

According to the culture device described in the eleventh aspect, a plurality of cells can be easily arranged in an aligned state, and each cell tissue can be easily handled after the cell tissues are separated from the substrate.

(Twelfth Aspect) In the culture device according to the tenth aspect, the at least one first opening has an elongated shape in which a length along a sliding direction of the first wall is shorter than a length along a direction orthogonal to the sliding direction when the first wall is viewed from a normal direction of a flat surface of the first wall. The at least one second opening has an elongated shape in which a length along a sliding direction of the second wall is shorter than a length along a direction orthogonal to the sliding direction when the second wall is viewed from a normal direction of a flat surface of the second wall.

According to the culture device described in the twelfth aspect, when a plurality of nozzles such as a multi-nozzle dispenser are inserted into the culture device at a time, it is easy to align the multi-nozzle dispenser as compared with the case where a plurality of openings are formed in the column direction. In addition, since the length along the sliding direction is shorter, the distance from the cell tissue to the edge of the first opening and the edge of the second opening can be reduced. This makes it possible to shorten the distance to slide the first wall and the second wall to separate the cell tissues from the first substrate and the second substrate.

It is contemplated that the embodiments disclosed herein may be appropriately combined and implemented as long as they are not technically contradictory to each other. It should be understood that the embodiments disclosed herein are illustrative and non-restrictive in all respects. The scope of the present invention is defined by the terms of the claims rather than the description of the embodiments above, and is intended to include any modifications within the scope and meaning equivalent to the terms of the claims.

It should be understood that the embodiments disclosed herein have been presented for the purpose of illustration and description but not limited in all aspects. It is intended that the scope of the present invention is not limited to the description above but defined by the scope of the claims and encompasses all modifications equivalent in meaning and scope to the claims.

What is claimed is:

1. A production method of at least one cell tissue, comprising:
linearly printing at least one cell between a first substrate and a second substrate in such a manner that both ends of the at least one cell are supported on the first substrate and the second substrate, respectively;
obtaining the at least one cell tissue by culturing the printed at least one cell;
filling a soluble liquid holding material that cures under a predetermined condition between the first substrate and the second substrate;
curing the filled holding material; and
separating the at least one cell tissue from at least one of the first substrate and the second substrate by cutting out the cured holding material from the at least one of the first substrate and the second substrate.

2. The production method according to claim 1, wherein
a first member having a flat plate shape and a second member having a flat plate shape are disposed between the first substrate and the second substrate in this order,
a flat surface of the first member is disposed to face the first substrate, and a flat surface of the second member is disposed to face the second substrate,
the first member is slidable along the flat surface of the first member,
the second member is slidable along the flat surface of the second member,
the separating includes separating the at least one cell tissue from the first substrate and the second substrate by sliding the first member and the second member.

3. The production method according to claim 2, wherein
the first member is formed with at least one first opening through which the at least one cell passes,
the second member is formed with at least one second opening through which the at least one cell passes, and
the printing includes printing the at least one cell through the at least one first opening and the at least one second opening.

4. The production method according to claim 1, wherein
the printing includes individually and linearly printing a plurality of cells,
the obtaining of the at least one cell tissue includes obtaining a plurality of cell tissues by culturing the plurality of cells, and
the production method further includes obtaining a plurality of blocks by dividing the holding material along an extending direction of the plurality of cell tissues held by the cured holding material.

5. The production method according to claim 1, further comprising:
dissolving the cut-out holding material.

6. The production method according to claim 4, further comprising:
disposing the plurality of blocks in such a manner that the extending directions of the plurality of cell tissues are aligned with each other; and
dissolving the holding material constituting each of the plurality of disposed blocks.

7. The production method according to claim 1, wherein
the filling of the holding material is performed after the at least one cell tissue is obtained.

8. A bioprinting culture device for culturing at least one cell to obtain at least one cell tissue, the culture device comprising:
a housing in which a first chamber with a first substrate for supporting the at least one cell disposed therein, a storage chamber configured to store a culture solution, and a second chamber with a second substrate for supporting the at least one cell disposed therein are provided in this order;
a replacement unit that replaces the solution stored in the storage chamber with a soluble liquid holding material that cures under a predetermined condition; and
a separation unit,
the at least one cell being printed linearly from the first chamber toward the second chamber,
the separation unit separating the at least one cell tissue stored in the storage chamber from at least one of the first substrate disposed in the first chamber and the second substrate disposed in the second chamber.

9. The culture device according to claim 8, wherein
the separation unit includes a first wall that is disposed in the housing to partition the first chamber and the storage chamber from each other, and a second wall that is disposed in the housing to partition the second chamber and the storage chamber from each other,
the first wall is slidable along a flat surface of the first wall,
the second wall is slidable along a flat surface of the second wall, and
the separation unit is configured to separate the first substrate and the second substrate from each of the at least one cell tissue by sliding the first wall and the second wall.

10. The culture device according to claim 9, wherein
the first wall is formed with at least one first opening through which the at least one cell passes, and
the second wall is formed with at least one second opening through which the at least one cell passes.

11. The culture device according to claim 10, wherein
the first wall is formed with a plurality of first openings arranged in two dimensions when the first wall is viewed from a normal direction of a flat surface of the first wall, and
the second wall is formed with a plurality of second openings arranged in two dimensions when the second wall is viewed from a normal direction of a flat surface of the second wall.

12. The culture device according to claim 10, wherein
the at least one first opening has an elongated shape in which a length along a sliding direction of the first wall is shorter than a length along a direction orthogonal to the sliding direction when the first wall is viewed from a normal direction of a flat surface of the first wall, and
the at least one second opening has an elongated shape in which a length along a sliding direction of the second wall is shorter than a length along a direction orthogonal to the sliding direction when the second wall is viewed from a normal direction of a flat surface of the second wall.
